(19) United States
(12) Patent Application Publication
(10) Pub. No.: US 2007/0275917 A1
(43) Pub. Date: Nov. 29, 2007

(54) Rna Capable of Suppressing Expression of Klf5 Gene

(75) Inventors: Ryozo Nagai, Tokyo (JP); Ichiro Manabe, Tokyo (JP); Atsushi Ishihara, Shizuoka (JP); Tsuneaki Tottori, Osaka (JP)

Correspondence Address: GREENBLUM & BERNSTEIN, P.L.C., 1950 ROLAND CLARKE PLACE, RESTON, VA, 20191, US

(73) Assignee: Kyowa Hakko Kogyo Co., Ltd, Tokyo (JP)

(21) Appl. No.: 10/565,997

(22) PCT Filed: Jul. 29, 2004
(86) PCT No.: PCT/JP04/11223

§ 371 Date: Aug. 14, 2006

(30) Foreign Application Priority Data
Jul. 29, 2003 (JP) 2003-202863
Mar. 16, 2004 (JP) 2004-075115

(51) Int. Cl. A61K 48/00; C07H 21/02

(52) U.S. Cl. 514/44; 435/320.1; 435/375; 536/24.5; 536/23.1

(57) ABSTRACT

An RNA capable of suppressing the expression of KLF5 gene, which comprises a sequence consisting of 15 to 30 contiguous nucleotides of KLF5 mRNA and a sequence complementary to the sequence, and which has been designed from the nucleotide sequence of Kruppel-like factor 5 (KLF5) cDNA. Specifically, a double-stranded RNA having a strand of a sequence shown in any one of SEQ ID NOS: 2 to 16 and a strand of a sequence complementary to the sequence, in which 2 uridylic acids are added to the 3′-terminus of each of the strands. By transfecting the RNA or a vector for expression of the RNA into cells, the expression of KLF5 gene in the cells can be suppressed. The RNA or a vector for expression of the RNA can be used as a therapeutic agent for cardiovascular disease or cancer.

### TECHNICAL FIELD

The present invention relates to RNA that is capable of suppressing the expression of KLF5 gene.

### BACKGROUND ART

The Kruppel-like factor (hereinafter abbreviated as KLF) family is a family of transcriptional factors, which is characterized in that it has zinc finger motifs at the C-terminus thereof, and examples thereof that have been known include KLF1, KLF2, KLF3, KLF4, KLF5, KLF6, KLF7, KLF8, KLF9, KLF10, KLF11, KLF12, KLF13, KLF14, KLF15, and KLF16. It has been reported that, in mammals, the KLF family plays an important role in differentiation of various types of tissues or cells, such as erythrocytes, vascular endothelial cells, smooth muscle, skin, or lymphocytes, and also in formation of the pathologic conditions of various types of diseases such as cancer, cardiovascular disease, hepatocirrhosis, renal disease, or immune-mediated disease (J. Biol. Chem., 276, 34355-34358, 2001; Genome Biol., 4, 206, 2003).

Among the KLF family members, KLF5 is also referred to as BTEB2 (basic transcriptional element binding protein 2) or IKLF (intestinal-enriched Kruppel-like factor). The expression of KLF5 in vascular smooth muscle is controlled at the development stage thereof. KLF5 is highly expressed in the vascular smooth muscle of a fetus, whereas its expression is not found in the vascular smooth muscle of a healthy adult. In addition, in the case of the smooth muscle of intima of a blood vessel regenerated after denudation by a balloon catheter, KLF5 is highly expressed. Also, in the smooth muscle of lesions due to arteriosclerosis or restenosis, KLF5 is expressed (Circulation, 102, 2528-2534, 2000).

The vascular smooth muscle cells of lesions such as an arteriosclerosis lesion or a restenosis site formed after percutaneous transluminal coronary angioplasty are activated. Such vascular smooth muscle cells exhibit the disappearance of myofilaments, stimulation of protein synthesis, growth ability, and migration ability. Thus, such vascular smooth muscle cells have been transformed, so as to have the same characteristics as those of the vascular smooth muscle of an embryo (embryonic type). In smooth muscle cells, 3 types of isoforms of a myosin heavy chain, such as SM1, SM2, and SMemb, are present. However, as the vascular smooth muscle is transformed to a fetal type, SM2 disappears, and induction of the expression of SMemb is observed. KLF5 binds to the transcriptional regulatory sequence of the SMemb gene, so as to activate the transcription thereof. Further, it has been reported that KLF5 activates the transcription of genes associated with the characteristics of a blood vessel or vascularization, such as platelet-derived growth factor A chain (hereinafter referred to as PDGF-A), transforming growth factor (TGF)-β, vascular endothelial growth factor (VEGF) receptor, inducible nitric oxide synthase (iNOS), plasminogen activator inhibitor (PAI)-1, or transcription factor Egr (early growth response)-1 (Nat. Med., 8, 856-863, 2002; Ann. N. Y. Acad. Sci., 947, 56-66, 2001).

Moreover, it has been reported that, in KLF5 gene hetero-knockout mice, the growth of vascular smooth muscle, endothelial proliferation, vascularization, formation of granulation in the adventitia of a blood vessel, cardiac hypertrophy, and the development of fibrotic cardiomyopathy, which are caused by physical loading on a cardiovascular system or angiotensin II, are significantly suppressed (Nat. Med., 8, 856-863, 2002).

Thus, the KLF5 gene is not only associated with transformation of smooth muscle, but it is also a transcriptional factor associated with formation of a broad range of pathologic conditions of the cardiovascular system. The expression level of the gene is extremely important for the expression of the functions thereof. KLF5 is associated with formation of the pathologic conditions of cardiovascular diseases such as arteriosclerosis or cardiac hypertrophy, or angiogenesis-related diseases such as cancer. Thus, it is anticipated that a pharmaceutical agent useful for the treatment or prevention of the aforementioned diseases be developed by suppressing the expression of the KLF gene. However, to date, a pharmaceutical agent for effectively suppressing the expression of the KLF family genes has not yet been known.

On the other hand, it has been reported that RNA interference (hereinafter referred to as RNAi) is a phenomenon whereby when double-stranded RNA having a sequence identical to that of a target gene is introduced into nematode, the expression of the target gene is specifically suppressed (Nature, 391, 806-811, 1998).

It is considered that the introduced double-stranded RNA is decomposed to double-stranded RNA having a length of 21 to 23 nucleotides, that a protein complex then binds to this short double-stranded RNA, that it recognizes mRNA having the same sequence and then cleaved it, and thus that RNAi takes place. Tuschl et al. have found that even when a double-stranded RNA having a length of 21 to 23 nucleotides is introduced into drosophila, instead of a long double-stranded RNA, the expression of a target gene is suppressed. This was named short interfering RNA (siRNA) (WO01/75164). When there is a mismatch between the sequence of siRNA and that of a target gene, the effect of suppressing expression is significantly reduced. The length consisting of 21 nucleotides brings on the highest effect. When such double-stranded RNA has a structure with protrusive termini obtained by adding nucleotides to 3′-termini of both strands, it provides a higher effect than that of double-stranded RNA having blunt ends (WO02/44321).

In the case of mammalian cells, when a long double-stranded RNA has been introduced, suppression of the expression of all genes and apoptosis have taken place as a result of the functions of virus defense mechanism, and thus suppression of a specific gene was impossible. However, it has been found that when siRNA having a length of 20 to 29 nucleotides is used, such a reaction does not take place, and that the expression of a specific gene can be suppressed. Among others, siRNA having 21 to 25 nucleotides has a high effect of suppressing expression (Nature, 411, 494-498, 2001; Nat. Rev. Genet., 3, 737-747, 2002; Mol. Cell, 10, 549-561, 2002; Nat. Biotechnol., 20, 497-500, 2002).

It has been reported that in RNAi, the effect of double-stranded RNA to suppress the expression of a target gene is significantly higher than that of single-stranded antisense RNA (Nature, 391, 806-811, 1998; Mol. Cell, 10, 549-561, 2002). In addition, it has also been reported that not only double-stranded RNA, but also single-stranded RNA forming a hairpin structure as a result of intramolecular hybridization, exhibits RNAi, as with siRNA (Proc. Natl. Acad. Sci. USA, 99, 6047-6052, 2002).

RNAi has been verified not only in vitro tests but also in in vivo tests. The effect of RNAi using siRNA with a length of 50 bp or less on fetal animals (WO02/132788) and the effect thereof on adult mice (WO03/10180) have been reported. Moreover, when siRNA is intravenously administered to a fetal mouse, the effect of suppressing expression was found in various organs such as kidney, spleen, lung, pancreas, and liver (Nat. Genet. 32, 107-108, 2002). Furthermore, it has been reported that when siRNA is directly administered to brain cells, it acts on them (Nat. Biotechnol., 20, 1006-1010, 2002). However, RNAi using siRNA on KLF5 or other KLF family genes has not yet been reported to date.

### DISCLOSURE OF THE INVENTION

It is an object of the present invention to find RNA that is capable of suppressing the expression of KLF5 gene. Such RNA suppresses the expression of the KLF gene, so as to inhibit the functions of KLF5 as a transcriptional factor. Thus, such RNA can be used as a therapeutic or preventive agent causing few side effects, which is used for diseases such as cardiovascular disease or cancer, of which diseases KLF5 is associated with formation of the pathologic conditions.

The present inventors have conducted intensive studies, and as a result, they have completed the following invention. That is to say, the present invention relates to the following (1) to (13):

(1) An RNA capable of suppressing the expression of KLF5 gene, which comprises a sequence consisting of 15 to 30 contiguous nucleotides of KLF5 mRNA and a sequence complementary to the sequence.

(2) The RNA according to (1), wherein the KLF5 mRNA is human KLF5 mRNA or mouse KLF5 mRNA.

(3) The RNA according to (1) or (2), wherein the RNA is a double-stranded RNA consisting of a strand of sequence consisting of 15 to 30 contiguous nucleotides of KLF5 mRNA and a strand of sequence complementary to the sequence, in which 1 to 6 nucleotides are added to the 3′-terminus of each of the strands.

(4) The RNA according to (1) or (2), wherein the RNA is an RNA forming a hairpin structure, which is obtained by ligating an RNA having a sequence consisting of 15 to 30 contiguous nucleotides of the KLF5 mRNA to an RNA having a sequence complementary to the sequence via a spacer oligonucleotide, and then adding 1 to 6 nucleotides to the 3′-terminus thereof.

(5) An RNA capable of suppressing the expression of KLF5 gene, which is selected from the group consisting of the following (a) to (c):

(a) a double-stranded RNA having a strand of a sequence shown in any one of SEQ ID NOS: 2 to 16 and a strand of a sequence complementary to the sequence, in which 2 to 4 uridylic acids or deoxythymidylic acids are added to the 3′-terminus of each of the strands;

(b) an RNA forming a hairpin structure, which is obtained by ligating an RNA having a sequence shown in any one of SEQ ID NOS: 2 to 16 to an RNA having a sequence complementary to the sequence via a spacer RNA that has 2 uridylic acids at the 5′-terminus thereof, and then adding 2 to 4 uridylic acids to the 3′-terminus thereof; and

(c) a double-stranded RNA consisting of a strand of a sequence shown in any one of SEQ ID NOS: 2 to 11 and a strand of a sequence complementary to the sequence, in which 2 uridylic acids are added to the 3′-terminus of each of the strands.

(6) A vector, which allows the RNA according to any one of (1) to (5) to be expressed.

(7) A method of suppressing the expression of KLF5 gene in cells by transfecting the RNA according to any one of (1) to (5) above or the vector according to (6) into the cells.

(8) A method of suppressing the expression of a gene whose transcription is activated by KLF5 in cells by transfecting the RNA according to any one of (1) to (5) or the vector according to (6) into the cells.

(9) The method according to (8), wherein the gene whose transcription is activated by KLF5 is platelet-derived growth factor A chain gene or a smooth muscle myosin heavy chain SMemb gene.

(10) A pharmaceutical composition, which comprises, as an active ingredient, the RNA according to any one of (1) to (5) or the vector according to (6).

(11) A pharmaceutical composition for inhibiting angiogenesis, which comprises, as an active ingredient, the RNA according to any one of (1) to (5) or the vector according to (6).

(12) A therapeutic or preventive agent for cardiovascular disease or cancer, which comprises, as an active ingredient, the RNA according to any one of (1) to (5) or the vector according to (6).

(13) The therapeutic or preventive agent according to (12) above, wherein the cardiovascular disease is arteriosclerosis, restenosis occurring after coronary intervention, or cardiac hypertrophy.

By using the RNA of the present invention, the expression of KLF5 gene and a gene whose transcription is activated by KLF5 can be suppressed. The expression of the KLF5 gene and the gene whose transcription is activated by KLF5 is suppressed by administration of the RNA of the present invention or a vector for expressing the above RNA, and as a result, the growth of smooth muscle or vascularization can be suppressed. Thus, the RNA of the present invention or the vector for expressing the above RNA can be used as an active ingredient of a therapeutic or preventive agent for cardiovascular diseases such as arteriosclerosis, restenosis occurring after coronary intervention or cardiac hypertrophy, or cancers.

1. RNA Capable of Suppressing the Expression of KLF5 Gene

The RNA of the present invention comprises a sequence consisting of 15 to 30, preferably 17 to 25, and more preferably 19 to 23 contiguous nucleotides of KLF5 mRNA (hereinafter referred to as sequence X) and a sequence complementary to the above sequence (hereinafter referred to as complementary sequence X′), and it is capable of suppressing the expression of KLF5 gene. Such RNA includes: (a) a double-stranded RNA which consists of a strand of sequence X (sense strand) and a strand of complementary sequence X′ (antisense strand), in which 1 to 6, and preferably 2 to 4 nucleotides are added to the 3′-terminus of each of the strands (hereinafter, an RNA having this structure is referred to as a siRNA), and which is capable of suppressing the expression of KLF5 gene; and (b) an RNA forming a hairpin structure, which is obtained by ligating an RNA having sequence X to an RNA having complementary sequence X′ via a spacer oligonucleotide, and then adding 1 to 6, and preferably 2 to 4 nucleotides to the 3′-terminus thereof (hereinafter, such RNA is referred to as a shRNA), and which is capable of suppressing the expression of KLF5 gene. Nucleotides added to such an RNA may be any one selected from guanine, adenine, cytosine, thymine, and uracil. Either RNA or DNA may be used as a nucleotide added to such an RNA. Uridylic acid (U) or deoxythymidylic acid (dT) is preferable. As a spacer oligonucleotide, an RNA consisting of 6 to 12 nucleotides is preferable. As a sequence at the 5′-terminus thereof, two nucleotides, UU, are preferable. An example of such a spacer oligonucleotide is an RNA consisting of the sequence UUCAAGAGA. Either one of two RNA portions that are ligated to each other via a spacer oligonucleotide may be on the 5′-terminal side.

As sequence X, any sequence may be used, as long as it consists of 15 to 30, preferably 17 to 25, and more preferably 19 to 23 contiguous nucleotides of KLF5 mRNA. A sequence consisting of 19 nucleotides designed by the method described in (1) below is most preferable. An RNA that has the above-described structure and is capable of suppressing the expression of KLF5 gene is included in the RNA of the present invention.

The RNA of the present invention can be obtained by transfecting an RNA having the aforementioned structure into cells in which KLF5 gene is expressed, measuring the expression of KLF5 gene, and selecting an RNA suppressing the expression of KLF5 gene.

(1) Design of Sequence X A sequence portion consisting of 21 nucleotides that begin with AA is extracted from the nucleotide sequence of KLF5 cDNA of an animal in which the gene expression is to be suppressed. The GC content of the extracted sequence is calculated, and several sequences having a GC content between 20% and 80%, preferably between 30% and 70%, and more preferably between 40% and 60%, are selected.

Such a sequence is preferably a sequence in a coding region, which is located 75 nucleotides or more downstream of a start codon. Information regarding the nucleotide sequence of KLF5 cDNA can be obtained from nucleotide sequence database such as GenBank. For example, with regard to sequence information, the sequence of mouse KLF5 cDNA can be obtained from GenBank Accession No. NM_{—}009769 (SEQ ID NO: 49), and the sequence of human KLF5 cDNA can be obtained from GenBank Accession No. AF287272 (SEQ ID NO: 50).

AA at the 5′-terminus is eliminated from the selected sequence, and T is then substituted with U in the sequence. The thus obtained sequence consisting of 19 nucleotides is defined as sequence X.

(2) Preparation of an RNA of the Present Invention

RNA can be prepared as follows based on sequence X selected in (1) above. A case where UU or dTdT are used as oligonucleotides to be added will be described below. However, RNA can also be prepared in the case that other nucleotides are used.

(a) Case of siRNA

An RNA having a sequence obtained by adding UU or dTdT to the 3′-terminus of sequence X, and an RNA having a sequence obtained by adding UU or dTdT to the 3′-terminus of complementary sequence X′, are prepared. Such two RNA portions can be prepared by chemical synthesis or in vitro transcription. Chemical synthesis can be carried out using a DNA synthesizer. Otherwise, it is also possible to ask some manufacturers such as Ambion, Japan Bio Services Co., Ltd., or QIAGEN, to carry out such chemical synthesis. The thus chemically synthesized two RNA portions comprising sequences complementary to each other are annealed, so as to prepare a double-stranded RNA consisting of a strand of a sequence X and a strand of a complementary sequence X′, in which UU or dTdT are added to the 3′-terminus of each of the strands. Annealing can be carried out by heating two RNA portions in a suitable buffer at a temperature between 90° C. and 95° C. for 1 to 5 minutes, and then cooling them to room temperature over 45 to 60 minutes.

RNA can be prepared via in vitro transcription as follows. First, the following DNA portions are prepared: (i) a DNA having the promoter sequence of T7 RNA polymerase (T7 primer); (ii) a DNA having a sequence obtained by substituting U with T in complementary sequence X′, adding AA at the 5′-terminus thereof, and further adding to the 3′-terminus thereof a sequence complementary to 8 nucleotides of the 3′-terminus of the T7 primer; and (iii) a DNA having a sequence obtained by substituting U with T in sequence X, adding AA at the 5′-terminus thereof, and further adding to the 3′-terminus thereof a sequence complementary to 8 nucleotides of the 3′-terminus of the T7 primer.

The T7 primer and the DNA of (ii) are annealed, and thereafter, they are converted to double-stranded DNA by a DNA polymerase reaction. Using the obtained double-stranded DNA as a template, an in vitro transcription reaction is carried out using T7 RNA polymerase, so as to synthesize an RNA having a sequence obtained by adding UU to the 3′-terminus of sequence X and adding a leader sequence to the 5′-terminus thereof. Likewise, the same reaction as mentioned above is carried out using the T7 primer and the DNA of (iii), so as to synthesize an RNA having a sequence obtained by adding UU to the 3′-terminus of complementary sequence X′ and adding a leader sequence to the 5′-terminus thereof.

The two reaction solutions are mixed, and such an in vitro transcription reaction is further continued, so that the two RNA portions having sequences complementary to each other are annealed. Thereafter, the double-stranded DNA used as a template and the leader sequence at the 5′-terminus of each RNA strand are digested with deoxyribonuclease and single-stranded RNA-specific ribonuclease, and then eliminated. The UU portion at the 3′-terminus of each RNA strand remains, without being digested.

The aforementioned reaction can be carried out using a kit such as Silencer·siRNA Construction Kit (manufactured by Ambion). DNA to be annealed with the T7 primer can be chemically synthesized using a DNA synthesizer. Moreover, it is also possible to ask some manufacturers such as Ambion, Japan Bio Services Co., Ltd., Hokkaido System Science Co., Ltd., or QIAGEN, to carry out such chemical synthesis.

(b) Case of shRNA

An RNA forming a hairpin structure obtained by ligating an RNA having sequence X to an RNA having complementary sequence X′ via a spacer oligonucleotide, and then adding 1 to 6, and preferably 2 to 4 nucleotides, to the 3′-terminus thereof, can be prepared by chemical synthesis using a DNA synthesizer. In addition, an siRNA expression vector described in Section 2 later is transfected into cells, so as to synthesize shRNA in the cells. This shRNA is converted to siRNA in the cells. When a vector is transfected into cells and such shRNA is synthesized therein, isolation of a shRNA and transfection thereof into cells described in (3) below are unnecessary. The expression of KLF5 gene may only be analyzed in the cells transfected with the vector.

(3) Suppression of the Expression of KLF5 Gene

The siRNA or shRNA prepared in (2) above is transfected into a cell line that expresses KLF5 gene. As a cell line, the cells of the same animal species as the KLF5 cDNA used as a base of the design of sequence X described in (1) above are used. Examples of a cell line that expresses KLF5 gene may include cell lines derived from smooth muscle, fibroblasts or vascular endothelial cells, such as the fetal mouse fibroblast cell line C3H/10T1/2 (ATCC No. CCL-226) or human umbilical cord vascular endothelial cells. Transfection of the RNA can be carried out using reagents for transfection into animal cells, such as Polyfect Transfection Reagent (manufactured by QIAGEN), TransMessenger Transfection Reagent, Oligofectamine Reagent (manufactured by Invitrogen), or Lipofectamine 2000 (manufactured by Invitrogen). These reagents are mixed with the RNA to form a complex, and the complex is then added to cells.

The expression of KLF5 gene in cells, which were transfected with the RNA of the present invention or an siRNA expression vector described later in Section 2, can be analyzed by RT-PCR. Total RNA is prepared from cells transfected with the RNA or the siRNA expression vector, and also from cells which were not transfected with the RNA or the siRNA expression vector. Thereafter, cDNA is synthesized from the RNA. Using the synthesized cDNA as a template, PCR is carried out with primers specific to KLF5 gene. The amount of an amplified product derived from KLF5 cDNA is quantified by agarose gel electrophoresis, thereby measuring the expression level of KLF5 gene. An RNA that was transfected into cells in which the expression level of the KLF5 gene is lower than the expression level of the KLF5 gene in cells which were not transfected with the RNA or the siRNA expression vector, is selected as an RNA capable of suppressing the expression of KLF5 gene.

An example of the thus selected RNA capable of suppressing the expression of KLF5 gene is a double-stranded RNA consisting of a strand of a sequence shown in any one of SEQ ID NOS: 2 to 11 and a strand of a sequence complementary to the above sequence, in which two uridylic acids are added to the 3′-terminus of each of the strands. This RNA is designed based on the sequence of mouse cDNA, and it suppresses the expression of mouse KLF5 gene. Among such sequences, sequences shown in SEQ ID NOS: 4, 8, and 10, are shared by mouse KLF5 mRNA and human KLF5 mRNA. Thus, a double-stranded RNA consisting of a strand of a sequence shown in any one of SEQ ID NOS: 4, 8, and 10, and a strand of a sequence complementary to the above sequence, in which two uridylic acids are added to the 3′-terminus of each of the strands, is capable of suppressing not only the mouse KLF5 gene but also the human KLF5 gene.

The KLF5 cDNA of a certain animal species “A” used as a base for the design of sequence X described in (1) above is aligned with the KLF5 cDNA of a different animal species “B” based on the sequence homology, so as to obtain sequence Y of the animal species “B” that corresponds to sequence X selected in the animal species “A”. When an RNA capable of suppressing the expression of KLF5 gene of the animal species “A” is obtained by the aforementioned method, an RNA obtained by substituting sequence X region and complementary sequence X′ region with sequence Y and complementary sequence Y′ in the RNA, respectively, is considered to be capable of suppressing KLF5 gene of the animal species “B”.

For example, a double-stranded RNA consisting of a strand of a sequence shown in any one of SEQ ID NOS: 2, 3, 7, 9, and 11, based on the sequence of mouse KLF5 cDNA, and a strand of a sequence complementary to the above sequence, in which two uridylic acids are added to the 3′-terminus of each of the strands, is capable of suppressing the expression of mouse KLF5 gene. Accordingly, a double-stranded RNA consisting of a strand of a sequence shown in any one of SEQ ID NOS: 12 to 16, which are the corresponding sequences of human KLF5 cDNA, and a strand of a sequence complementary to the above sequence, in which two uridylic acids are added to the 3′-terminus of each of the strands, is considered to be capable of suppressing the expression of the human KLF5 gene.

2. Vector for Expressing RNA Capable of Suppressing the Expression of KLF5 Gene

(1) Plasmid Vector

A plasmid vector for expressing an RNA capable of suppressing the expression of KLF5 gene is transfected into cultured cells or cells in a living body, so that the above RNA can be generated in the cells, thereby suppressing the expression of KLF5 gene in the transfected cells. This vector can be produced by inserting downstream of the promoter of an siRNA expression vector such as a plasmid vector used for animal cells comprising an RNA polymerase III promoter such as U6 promoter or H1 promoter, a DNA obtained by ligating sequence X selected in Section 1 to complementary sequence X′ thereof (wherein, in each sequence, U is substituted with T) via a spacer sequence having TT at the 5′-terminus thereof, which comprises a sequence portion consisting of 4 to 6 Ts acting as an RNA polymerase III terminator at the 3′-terminus thereof (hereinafter referred to as DNA used for KLF5 siRNA). As a spacer sequence, a sequence consisting of 6 to 12 nucleotides and having TT at the 5′-terminus thereof is preferable. An example of such a sequence is TTCAAGAGA. Either one of sequence X and complementary sequence X′ may be on the 5′-terminal side. Examples of an siRNA expression vector may include pSilencer 1.0-U6 (manufactured by Ambion), pSilencer 3.0 (manufactured by Ambion), pSUPER (manufactured by OligoEngine), and pSIREN-DNR (manufactured by BD Biosciences Clontech).

In cells transfected with a recombinant vector constructed by insertion of the aforementioned DNA used for KLF5 siRNA, the shRNA described in Section 1. (1) is synthesized by an RNA polymerase III reaction from U6 promoter. Thereafter, this shRNA is cleaved in the cells and then converted to a siRNA. Such a recombinant vector can be transfected into cells by the calcium phosphate method (Japanese Published Unexamined Patent Application no. 227075/90), the lipofection method (Proc. Natl. Acad. Sci. USA, 84, 7413-7417, 1987), or the like, as with common transfection of a vector into animal cells.

(2) Viral Vector

As an siRNA expression vector, a siRNA expression vector utilizing viral vectors such as a retrovirus vector, lentivirus vector, or adenovirus vector, can be used. Examples of an siRNA expression vector utilizing such viral vectors may include pSUPER.retro (manufactured by OligoEngine), pSIREN-RetroQ (BD Biosciences-Clontech), and a vector described in the publication (Proc. Natl. Acad. Sci USA, 100 1844-1848, 2003; Nat. Genet., 33, 401-406, 2003).

The same DNA used for KLF5 siRNA as mentioned above is inserted into an siRNA expression vector utilizing a viral vector, so as to prepare a recombinant vector. Thereafter, the obtained vector is transfected into packaging cells which are selected based on the utilized viral vector, thereby producing a recombinant virus comprising the above recombinant vector. Such a recombinant vector can be transfected into packaging cells by the calcium phosphate method or the lipofection method, as described above. The obtained recombinant virus is allowed to contact with cells, and the cells are thereby infected therewith, so as to transfect a recombinant vector into the cells. As a result, the shRNA described in Section 1. (1) is synthesized, and the synthesized shRNA is then cleaved in the cells, so that the shRNA can be converted to a siRNA capable of suppressing the expression of KLF5 gene.

3. Use of RNA Capable of Suppressing the Expression of KLF5 Gene

(1) Suppression of the Expression of Gene Whose Transcription is Activated by KLF5

KLF5 acts as a transcription factor and activates the expression of various genes. When the expression of KLF5 gene is suppressed by an RNA capable of suppressing it, the expression of genes whose transcription is activated by KLF5 can also be suppressed. Examples of such a gene whose transcription is activated by KLF5 may include the genes such as SMemb, PDGF-A, TGF-β, a VEGF receptor, PAI-1, and Egr-1.

(2) Analysis of Functions of KLF5

An RNA capable of suppressing the expression of KLF5 gene is allowed to act on various types of cells, and a change in the characteristics of the cells or a change in the expression level of each gene are then examined, so as to analyze the functions of KLF5 in various types of cells. In addition, the above RNA is capable of suppressing the expression of KLF5 gene in animals that are at various development stages ranging from a fetus to an adult, and thus it becomes possible to clarify the functions of KLF5, which cannot be clarified only by the analysis of hetero-knockout mice.

4. Pharmaceutical Composition Comprising, as Active Ingredient, RNA or Vector of the Present Invention

The expression of KLF5 and the genes whose transcription is activated by KLF5 is suppressed by administration of an RNA that is specifically capable of suppressing the expression of KLF5 gene of the present invention or a vector for expressing the above RNA, so as to inhibit the growth of smooth muscle or the angiogenesis. Hence, the above RNA or vector can be used to treat or prevent cardiovascular diseases such as arteriosclerosis, restenosis occurring after coronary intervention or cardiac hypertrophy, or cancers.

When the RNA of the present invention or a vector for expressing the above RNA is used as a medicament, it can be administered singly. In general, however, it is preferable that such an RNA or a vector be mixed with pharmacologically acceptable additives (for example, a carrier, an excipient, a diluent, etc.), stabilizers, or pharmaceutically necessary components, and that it be provided in the form of a pharmaceutical preparation produced by any given method well known in the technical field of pharmaceutics. In the case of a viral vector, it is preferably administered in the form of a recombinant virus.

It is preferable that an administration route that is most effective for treatment be used. Examples of an administration route used herein may include: parenteral administration routes such as intraoral administration, tracheobronchial administration, intrarectal administration, subcutaneous administration, intramuscular administration, or intravenous administration; and an oral administration route. Preferred administration routes include intravenous administration and intramuscular administration. An example of a pharmaceutical preparation suitable for intravenous administration or intramuscular administration is an injection.

When the RNA of the present invention or a vector for expressing the above RNA is molded in the form of an injection, examples of a carrier used herein may include: diluents such as water, ethyl alcohol, macrogol, propylene glycol, citric acid, acetic acid, phosphoric acid, lactic acid, sodium lactate, sulfuric acid, or sodium hydroxide; pH adjusters and buffers such as sodium citrate, sodium acetate, or sodium phosphate; and stabilizers such as sodium pyrosulfite, ethylenediaminetetraacetic acid, thioglycolic acid, or thiolactic acid. In such a case, common salt, glucose, mannitol, or glycerin may be contained in the pharmaceutical preparation, at an amount sufficient for preparation of an isotonic solution. Examples of a stabilizer may include: monosaccharides such as glucose; disaccharides such as saccharose or maltose; sugar alcohols such as mannitol or sorbitol; neutral salts such as sodium chloride; amino acids such as glycine; nonionic surfactants such as polyethylene glycol, a polyoxyethylene-polyoxypropylene copolymer (Pluronic), or polyoxyethylene sorbitan fatty acid ester (Tween); and human albumin. In addition, in order to promote incorporation of the RNA of the present invention or a vector expressing the above RNA into cells, a liposome comprising the above RNA or vector may be prepared and used.

### BRIEF DESCRIPTION OF THE DRAWINGS

FIG. 1 shows suppression of the expression of KLF5 gene by KLF5 gene-specific siRNA. From the left, the figure shows a 100-bp marker, and the results obtained by measurement by PCR on cells which were not transfected with a siRNA, and cells which were transfected with each of SEAP-siRNA, siRNA No. 2, siRNA No. 3, siRNA No. 4, siRNA No. 5, and siRNA No. 6. KLF5 represents the position of an amplified product derived from KLF5 mRNA, and 18S represents the position of an amplified product derived from 18S rRNA.

FIG. 2 shows suppression of the expression of KLF5 gene by KLF5 gene-specific siRNA. From the left, the figure shows a 100-bp marker, and the results obtained by measurement by PCR on cells which were not transfected with a siRNA, and cells which were transfected with each of SEAP-siRNA, siRNA No. 7, siRNA No. 8, siRNA No. 9, siRNA No. 10, siRNA No. 11, siRNA No. 4, and siRNA No. 1. KLF5 represents the position of an amplified product derived from KLF5 mRNA, and 18S represents the position of an amplified product derived from 18S rRNA.

FIG. 3 shows suppression of the expression of PDGF-A gene by KLF5 gene-specific siRNA. From the left, the figure shows a 100-bp marker, and the results obtained by measurement by PCR on cells which were not transfected with a siRNA, and cells which were transfected with each of SEAP-siRNA, siRNA No. 7, siRNA No. 8, siRNA No.9, siRNA No.10, siRNA No.11, siRNA No.4, and siRNA No.1. PDGF-A represents the position of an amplified product derived from PDGF-A mRNA, and 18S represents the position of an amplified product derived from 18S rRNA.

FIG. 4 shows suppression of the expression of SMemb gene by KLF5 gene-specific siRNA. From the left, the figure shows a 100-bp marker, and the results obtained by measurement by PCR on cells which were not transfected with a siRNA, and cells which were transfected with each of SEAP-siRNA, siRNA No. 7, siRNA No. 8, siRNA No. 9, siRNA No. 10, siRNA No. 11, siRNA No. 4, and siRNA No. 1. SMemb represents the position of an amplified product derived from SMemb mRNA, and 18S represents the position of an amplified product derived from 18S rRNA.

FIG. 5 shows that KLF5 gene-specific siRNA does not suppress the expression of SRF gene. From the left, the figure shows the results obtained by measurement by PCR on cells which were not transfected with a siRNA, and cells which were transfected with each of SEAP-siRNA, siRNA No. 1, siRNA No. 4, siRNA No. 7, siRNA No. 9, and siRNA No. 10. SRF represents the position of an amplified product derived from SRF mRNA, and 18S represents the position of an amplified product derived from 18S rRNA.

FIG. 6 shows suppression of the expression of human KLF5 gene by siRNA No. 4. From the left, the figure shows a 100-bp marker, and the results obtained by measurement by PCR on cells which were not transfected with a siRNA, and cells which were transfected with either SEAP-siRNA or siRNA No. 4. KLF represents the position of an amplified product derived from KLF5 mRNA, and 18S represents the position of an amplified product derived from 18S rRNA.

FIG. 7 shows inhibition of the migration of vascular endothelial cells by siRNA No. 4. The horizontal axis represents time (hours), and the longitudinal axis represents the number of cells migrated. ● represents the results regarding cells transfected with siRNA No. 4, and ▪ represents the results regarding cells transfected with SEAP-siRNA. The error bar represents a standard deviation of 4 cases.

FIG. 8 shows the antitumor effect of siRNA No. 4. The horizontal axis represents time (number of days), and the longitudinal axis represents tumor volume (mm³). ◯ represents the tumor volume of a mouse to which KLF5 siRNA No. 4 has been administered, and ▪ represents the tumor volume of a mouse to which SEAP-siRNA has been administered.

### BEST MODE FOR CARRYING OUT THE INVENTION

The present invention will be more specifically described in the following examples. However, the present invention is not limited by these examples.

### EXAMPLE 1

### Suppression of the Expression of KLF5 Gene by siRNA

(1) Preparation of siRNA

As the sequence of siRNA which can suppress expression of KLF5 gene, from the sequence of mouse KLF5 cDNA (GenBank Registration No. NM_{—}009769; SEQ ID NO: 49), 11 sequence portions that satisfy the following 2 requirements were selected: (a) a sequence consisting of 21 nucleotides that begin with AA; and (b) a GC content between 20% and 80%. Such sequence portions were preferably selected from sequence portions, which are located in a coding region (the sequence portion at nucleotides 167-1507 of SEQ ID NO: 49) and further located 75 nucleotides or more downstream of a start codon (the sequence portion at nucleotides 167-169 of SEQ ID NO: 49), and which have a GC content between 40% and 60%. The positions of the selected sequences in SEQ ID NO: 49 and GC contents thereof are shown in Table 1. Sequences obtained by substituting T with U in 19 nucleotides excluding AA at the 5′-terminus thereof in the selected sequences were shown in SEQ ID NOS: 1 to 11.

TABLE 1

| | | | | SEQ | |
|---|---|---|---|---|---|
| | Sequence | GC | | ID | siRNA |
| Sequence selected | position | content | Produced RNA sequence | No. | No. |
| AACATGAACGTCTTCCTCCCT | 537-556 | 48% | CAUGAACGUCUUCCUCCCUTT | 17 | No. 1 |
| | | (10/21) | AGGGAGGAAGACGUUCAUGTT | 18 | |
| AAATTTACCTGCCACTCTGCC | 1156-1176 | 48% | AUUUACCUGCCACUCUGCCUU | 19 | No. 2 |
| | | (10/21) | GGCAGAGUGGCAGGUAAAUUU | 20 | |
| AAGGAGTAACCCGGATCTGGA | 1216-1236 | 52% | GGAGUAACCCGGAUCUGGAUU | 21 | No. 3 |
| | | (11/21) | UCCAGAUCCGGGUUACUCCUU | 22 | |
| AAAAGCTCACCTGAGGACTCA | 1303-1323 | 48% | AAGCUCACCUGAGGACUCAUU | 23 | No. 4 |
| | | (10/21) | UGAGUCCUCAGGUGAGCUUUU | 24 | |
| AATCCCCAGACCGTCCATGCC | 151-171 | 62% | UCCCCAGACCGUCCAUGCCUU | 25 | No. 5 |
| | | (13/21) | GGCAUGGACGGUCUGGGGGUU | 26 | |
| AACGCTGCGCCCACCCGCCTG | 1515-1535 | 76% | CGCUGCGCCCACCCGCCUGUU | 27 | No. 6 |
| | | (16/21) | CAGGCGGGUGGGCGCAGCGUU | 28 | |
| AAATGGAGAAGTATCTGACCC | 405-425 | 43% | AUGGAGAAGUAUCUGACCCUU | 29 | No. 7 |
| | | (9/21) | GGGUCAGAUACUUCUCCAUUU | 30 | |
| AAAGTATAGACGAGACAGTGC | 463-483 | 43% | AGUAUAGACGAGACAGUGCUU | 31 | No. 8 |
| | | (9/21) | GCACUGUCUCGUCUAUACUUU | 32 | |
| AAACCAGACGGCAGTAATGGA | 874-894 | 48% | ACCAGACGGCAGUAAUGGAUU | 33 | No. 9 |
| | | (10/21) | UCCAUUACUGCCGUCUGGCUU | 34 | |
| AAGCTCAGAGCCTGGAAGTCC | 2048-2068 | 57% | GCUCAGAGCCUGGAAGUCCUU | 35 | No. 10 |
| | | (12/21) | GGACUUCCAGGCUCUGAGCUU | 36 | |
| AAGCCGTTCCAGTGCATGGTG | 1424-1444 | 57% | GCCGUUCCAGUGCAUGGUGUU | 37 | No. 11 |
| | | (12/21) | CACCAUGCACUGGAACGGCUU | 38 | |

11 types of double-stranded RNA (hereinafter referred to as siRNA Nos. 1 to 11), which have a sequence shown in any one of SEQ ID NOS: 1 to 11 and a sequence complementary thereto, wherein UU or dTdT are added to the 3′-terminus of each of sequences, were prepared as follows. The sequences of the sense strand and antisense strand of each of siRNA Nos. 1 to 11 are shown in Table 1 (SEQ ID NOS: 17 to 38). siRNA No. 1 was prepared as follows. Namely, chemical synthesis of two RNAs shown in SEQ ID NOS: 17 and 18 was carried out by Japan Bio Services, Co., Ltd., and the thus obtained RNAs were annealed with each other. SiRNA Nos. 2 to 11 were prepared by in vitro transcription using Silencer™ siRNA Construction Kit (manufactured by Ambion). DNA used to produce a template used for the in vitro transcription was prepared by Hokkaido System Science Co., Ltd. Moreover, siRNA having sequences shown in SEQ ID NOS: 39 and 40, which is capable of suppressing the expression of secretory alkaline phosphatase (SEAP) gene (hereinafter referred to as SEAP-siRNA), was prepared by in vitro transcription using Silencer siRNA Construction Kit, based on the publications (Nat. Genet., 32, 107-108, 2002; and US patent Application Laid-Open No. 2002/0132788). This siRNA was used as a control siRNA.

(2) Suppression of the Expression of KLF5 Gene by siRNA

The fetal mouse fibroblast cell line C3H/10T1/2 (obtained from American Type Culture Collection (ATCC); ATCC No. CCL-226) was inoculated in a 6-well plate (manufactured by Corning), resulting in a concentration of 4×10⁵ cells per well. Thereafter, 10 μl of a reagent for transfection into cells, Polyfect (manufactured by QIAGEN), was added to each of 1.5 μg of siRNA No. 2, No. 3, No. 4, No. 5 and No. 6, and SEAP-siRNA, and then mixed. Each of the obtained mixtures was retained at room temperature for 5 to 10 minutes, and it was then added to each well. The cells were incubated in the presence of 5% CO₂ at 37° C. for 48 to 72 hours, so that each siRNA was transfected into the cells.

Suppression of the expression of KLF5 gene by the siRNA was confirmed by RT-PCR as shown below. After the incubation, RNA was isolated from the recovered cells, using a cell lysate-homogenizing kit QIAshredder (manufactured by QIAGEN) and a total RNA purification kit RNeasy (manufactured by QIAGEN). The isolated RNA was dissolved in 30 to 50 μl of water for injection (Otsuka distilled water, manufactured by Otsuka Pharmaceutical Co., Ltd.), and cDNA was then synthesized by a reverse transcription reaction. The reverse transcription reaction was carried out at 42° C. for 1.5 hours, using a reaction solution prepared by mixing the aforementioned RNA solution (containing 1.0 μg of RNA) with a solution containing 2.5 μl of 5× buffer, 2.0 μl of 0.1 mol/L dithiothreitol (DTT), 1.0 μl of 20 mmol/L dNTP (manufactured by Roche), 2.0 μl of a 50 μmol/L random primer (manufactured by Takara Shuzo Co., Ltd.), 1.0 μl of nuclease inhibitor SUPERase-In (manufactured by Ambion), and 1.0 μl of PowerScript reverse transcriptase (manufactured by Clontech), and then adding the water for injection thereto to a total amount of 18 μl. As such 5× buffer and DTT, those attached with the PowerScript reverse transcriptase were used.

Two DNAs each having the sequence shown in SEQ ID NO: 41 or 42 were chemically synthesized. These DNAs were used as mouse KLF5 gene-specific forward primer and reverse primer. By carrying out PCR using these primers, a 161-bp fragment corresponding to a sequence portion from nucleotides 1268 to 1428 of SEQ ID NO: 49 is amplified from KLF5 cDNA.

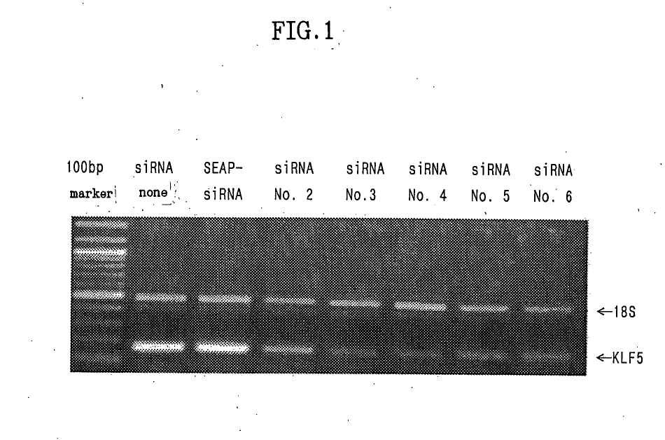

There was prepared 25 μl of a PCR reaction solution consisting of 2.5 μl of 10×PCR buffer, 2.0 μl of 2.5 mmol/L dNTP (manufactured by Roche), 2.0 μl of a 5 μmol/L forward primer, 2.0 μl of a 5 μmol/L reverse primer, 0.125 μl of HotStarTaq DNA polymerase (manufactured by QIAGEN; 5 units/μL), 2 μl of a 18S rRNA-specific primer (QuantumRNA Classic 18S Internal Standard; manufactured by Ambion), 13.375 μl of the water for injection, and 1.0 μl of cDNA. The prepared reaction solution was retained at 95° C. for 15 minutes. Thereafter, a reaction consisting of heat denaturation at 94° C. for 30 seconds, annealing at 53° C. for 30 seconds, and elongation at 72° C. for 40 seconds, was defined as 1 cycle, and PCR of 28 cycles was carried out. Thereafter, the reaction product was retained at 72° C. for 10 minutes. As a 10×PCR buffer, a buffer attached with the HotStarTaq DNA polymerase was used. After completion of the reaction, the obtained reaction solution was subjected to 0.8% agarose gel electrophoresis, so as to detect an amplified product (161 bp) derived from KLF5 mRNA. The amount of the obtained amplified product was compared with the amount of an amplified product in cells which were not transfected with a siRNA. An amplified product (488 bp) derived from 18S rRNA was used as an internal standard. As shown in FIG. 1, SEAP-siRNA used as a control did not suppress the expression of KLF5 gene, whereas it was confirmed that KLF5 gene-specific siRNA No. 2, No. 3, No. 4, No. 5, and No. 6, suppressed the expression of KLF5 gene. Among them, siRNA No. 3 and siRNA No. 4 strongly suppressed the expression of KLF5 gene.

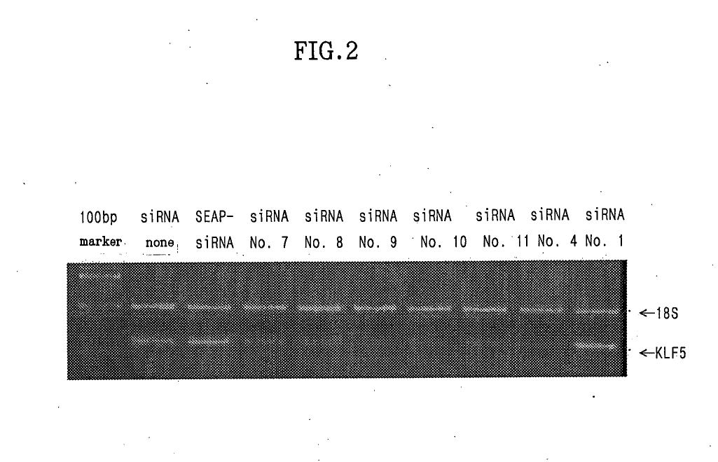

Using siRNA No. 1, No. 4, No. 7, No. 8, No. 9, No. 10, and No. 11, as KLF5 gene-specific siRNA, such siRNA was transfected into C3H/10T1/2 cells and the expression of KLF5 gene was then analyzed by RT-PCR, as described above. As shown in FIG. 2, SEAP-siRNA used as a control did not suppress the expression of KLF5 gene, whereas it was confirmed that KLF5 gene-specific siRNA No. 4, No. 7, No. 8, No. 9, No. 10, and No. 11, suppressed the expression of KLF5 gene. Among them, siRNA No. 4, siRNA No. 7, siRNA No. 9, and siRNA No. 10, strongly suppressed the expression of KLF5 gene. Although siRNA No. 1 is specific to the KLF5 gene, it did not suppress the expression thereof.

### EXAMPLE 2

### Suppression of the Expression of Genes Whose Transcription is Activated by KLF5, by KLF5 Gene-Specific siRNA

(1) Suppression of the Expression of PDGF-A Gene

As KLF5 gene-specific siRNA, each of siRNA No. 1, No. 4, No. 7, No. 8, No. 9, No. 10, and No. 11, was transfected into C3H/10T1/2 cells. Thereafter, the expression of PDGF-A gene whose transcription is activated by KLF5 was analyzed by RT-PCR.

Each siRNA was transfected into C3H/10T1/2 cells and cDNA was then prepared in the same manner as in Example 1(2). Two DNAs each having the sequence shown in SEQ ID NO: 43 or 44 were chemically synthesized. These DNAs were used as PDGF-A gene-specific forward primer and reverse primer. By carrying out PCR using these primers, a 403-bp fragment is amplified from PDGF-A cDNA. The PDGF-A gene-specific forward primer and reverse primer were used instead of the KLF5 gene-specific forward primer and reverse primer, and the expression of PDGF-A gene was analyzed in the same manner as in the analysis of the expression of KLF5 gene of Example 1(2). The PCR reaction solution was retained at 95° C. for 15 minutes. Thereafter, a reaction consisting of heat denaturation at 94° C. for 30 seconds, annealing at 53° C. for 30 seconds, and elongation at 72° C. for 40 seconds, was defined as 1 cycle, and PCR of 26 cycles was carried out. Thereafter, the reaction product was retained at 72° C. for 10 minutes. After completion of the reaction, the obtained reaction solution was subjected to 1% agarose gel electrophoresis.

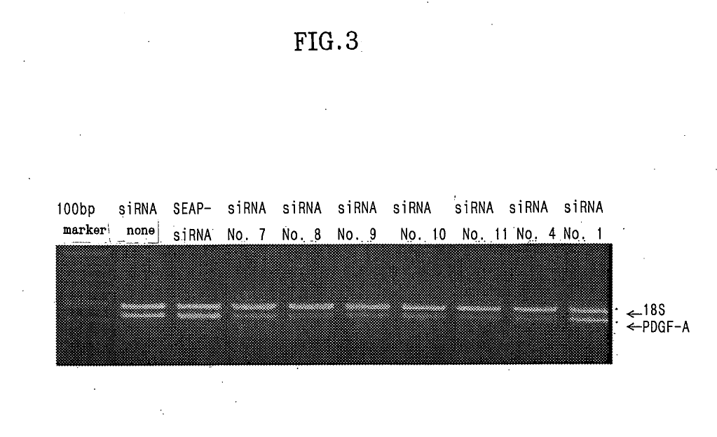

As shown in FIG. 3, SEAP-siRNA used as a control did not suppress the expression of PDGF-A gene, whereas it was confirmed that KLF5 gene-specific siRNA No. 4, No. 7, No. 8, No. 9, No. 10, and No. 11 suppressed also the expression of PDGF-A gene whose transcription is activated by KLF5. Among them, siRNA No. 4, siRNA No. 7, siRNA No. 9, and siRNA No. 10, strongly suppressed the expression of PDGF-A gene. Although siRNA No. 1 is specific to the KLF5 gene, it did not suppress the expression thereof.

(2) Suppression of the Expression of SMemb Gene

As KLF5 gene-specific siRNA, each of siRNA No. 1, No. 4, No. 7, No. 8, No. 9, No. 10, and No. 11, was transfected into C3H/10T1/2 cells. Thereafter, the expression of SMemb gene whose transcription is activated by KLF5 was analyzed by RT-PCR.

Each siRNA was transfected into C3H/10T1/2 cells and cDNA was then prepared in the same manner as in Example 1(2). Two DNAs each having the sequence shown in SEQ ID NO: 45 or 46 were chemically synthesized. These DNAs were used as SMemb gene-specific forward primer and reverse primer. By carrying out PCR using these primers, a 235-bp fragment is amplified from SMemb cDNA. The SMemb gene-specific forward primer and reverse primer were used instead of the KLF5 gene-specific forward primer and reverse primer, and the expression of SMemb gene was analyzed in the same manner as in the analysis of the expression of KLF5 gene of Example 1(2). The PCR reaction solution was retained at 95° C. for 15 minutes. Thereafter, a reaction consisting of heat denaturation at 94° C. for 30 seconds, annealing at 53° C. for 30 seconds, and elongation at 72° C. for 40 seconds, was defined as 1 cycle, and PCR of 26 cycles was carried out. Thereafter, the reaction product was retained at 72° C. for 10 minutes. After completion of the reaction, the obtained reaction solution was subjected to 1% agarose gel electrophoresis.

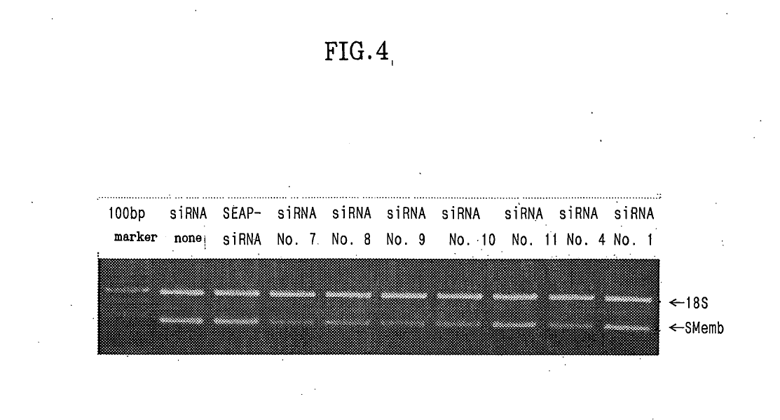

As shown in FIG. 4, SEAP-siRNA used as a control did not suppress the expression of SMemb gene, whereas it was confirmed that KLF5 gene-specific siRNA No. 4, No. 7, No. 8, No. 9, No. 10, and No. 11 suppressed also the expression of SMemb gene whose transcription is activated by KLF5. Among them, siRNA No. 4, siRNA No. 7, siRNA No. 9, and siRNA No. 10, strongly suppressed the expression of SMemb gene. Although siRNA No. 1 is specific to the KLF5 gene, it did not suppress the expression thereof.

(3) Specificity of Suppression of Gene Expression by KLF5 Gene-Specific siRNA

KLF5 gene-specific siRNA was transfected into C3H/10T1/2 cells, and the expression of serum response factor (SRF) gene was then analyzed by RT-PCR, so as to verify that suppression of gene expression by KLF5 gene-specific siRNA is specific to the KLF5 gene or genes whose transcription is activated by KLF5. The SRF gene is a transcription factor gene that is highly expressed in smooth muscle cells, and it is not a gene whose transcription is activated by KLF5.

As KLF5 gene-specific siRNA, each of siRNA No. 1, No. 4, No. 7, No. 9, and No. 10 were used. Each siRNA was transfected into C3H/10T1/2 cells and the gene expression was then analyzed by RT-PCR in the same manner as in Example 1(2). Two DNAs each having the sequence shown in SEQ ID NO: 47 or 48 were chemically synthesized. These DNAs were used as SRF gene-specific forward primer and reverse primer. By carrying out PCR using these primers, a 519-bp fragment is amplified from SRF cDNA. The SRF gene-specific forward primer and reverse primer were used instead of the KLF5 gene-specific forward primer and reverse primer, and the expression of SRF gene was analyzed in the same manner as in the analysis of the expression of KLF5 gene of Example 1(2). The PCR reaction solution was retained at 95° C. for 15 minutes. Thereafter, a reaction consisting of heat denaturation at 94° C. for 30 seconds, annealing at 53° C. for 30 seconds, and elongation at 72° C. for 40 seconds, was defined as 1 cycle, and PCR of 26 cycles was carried out. Thereafter, the reaction product was retained at 72° C. for 10 minutes. After completion of the reaction, the obtained reaction solution was subjected to 1.2% agarose gel electrophoresis.

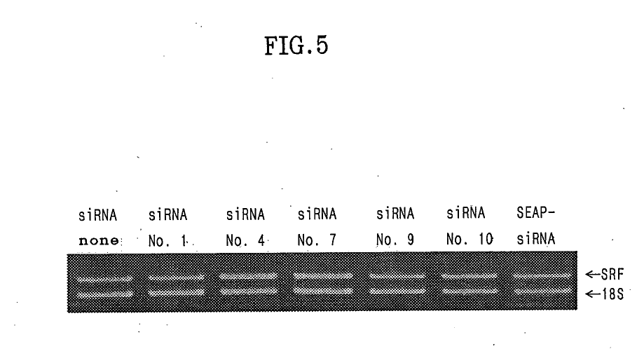

As shown in FIG. 5, as in the case of SEAP-siRNA used as a control, all of KLF5 gene-specific siRNA No. 1, No. 4, No. 7, No. 9, and No. 10 did not suppress the expression of SRF gene. Therefore, it became clear that KLF5 gene-specific siRNA does not suppress the expression of all such genes non-specifically, but that it specifically suppresses the expression of KLF5 gene and genes whose transcription is activated by KLF5.

### EXAMPLE 3

### Suppression of the Expression of Human KLF5 Gene by siRNA

The siRNA No. 4 prepared in Example 1 is siRNA produced based on the sequence (AAAAGCTCACCTGAGGACTCA) at nucleotides 1303 to 1323 of the nucleotide sequence (SEQ ID NO: 49) of mouse KLF5 cDNA. The siRNA No. 4 strongly suppressed the expression of mouse KLF5 gene in C3H/10T1/2 cells. However, this sequence, AAAAGCTCACCTGAGGACTCA, also exists at nucleotides 1481 to 1501 of the nucleotide sequence (SEQ ID NO: 50) of human KLF5 cDNA. Thus, it is anticipated that siRNA No. 4 inhibit not only the expression of mouse KLF5 gene but also that of human KLF5 gene. As described below, it was confirmed that siRNA No. 4 strongly suppressed the expression of human KLF5 gene.

Human umbilical cord venous endothelial cells (HUVEC; procured from Sanko Junyaku Co., Ltd.; Production No. CC-2517) were inoculated into a 6-cm dish (Corning), resulting in a concentration of approximately 3×10⁵ cells. 10 μl of a cell transfection reagent (Lipofectamine 2000, manufactured by Invitrogen) was added to each of 200 μmol siRNA No. 4 and SEAP-siRNA, and then mixed. The obtained mixture was retained at room temperature for 20 minutes, and the total amount thereof was then added to each dish. Thereafter, the cells were incubated in the presence of 5% CO₂ at 37° C. for 24 hours, so as to transfect each siRNA into the cells.

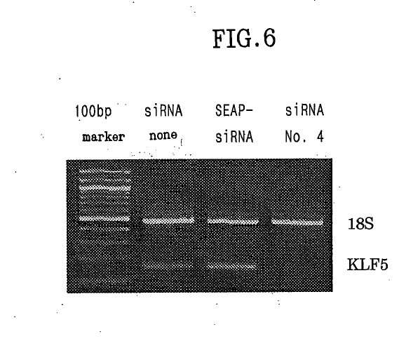

Thereafter, RNA was isolated from the cells by the same method as described in Example 1(2), and suppression of the expression of human KLF5 gene was then examined by RT-PCR. DNAs each having the sequence shown in SEQ ID NO: 41 or 42 which were used in Example 1 were used as KLF5 gene-specific forward primer and reverse primer, respectively. By carrying out PCR using these primers, a 161-bp fragment corresponding to a sequence portion from nucleotides 1446 to 1606 of SEQ ID NO: 50 is amplified from human KLF5 cDNA. After completion of the reaction, the obtained reaction solution was subjected to 0.8% agarose gel electrophoresis, so as to detect an amplified product (161 bp) derived from KLF5 mRNA. The amount of the obtained amplified product was compared with the amount of an amplified product in cells which were not transfected with a siRNA. An amplified product (488 bp) derived from 18S rRNA was used as an internal standard. As shown in FIG. 6, SEAP-siRNA used as a control did not suppress the expression of KLF5 gene, whereas KLF5 gene-specific siRNA No. 4 suppressed the expression of KLF5 gene in the human umbilical cord venous endothelial cells. Thus, it was confirmed that siRNA No. 4 could strongly suppress not only mouse KLF5 gene but also a human KLF5 gene.

With regard to siRNA Nos. 2 to 4 and 7 to 11 that suppressed the expression of a mouse KLF5 gene in Example 1, Table 2 shows: sequence portions consisting of 21 nucleotides on mouse KLF5 cDNA used as bases for the design of siRNA Nos. 2 to 4 and 7 to 11, and the positions thereof on SEQ ID NO: 49; sequence portions consisting of 21 nucleotides on human cDNA corresponding to the above mouse sequences, and the positions thereof on SEQ ID NO: 50; and SEQ ID NOS indicating RNA sequences obtained by eliminating AA at the 5′-terminus from the above human sequences. Since siRNA No. 5 and No. 6 were based on the sequence of a non-coding region, human sequences corresponding thereto were not indicated. It is considered that double-stranded RNA produced based on these human sequences also suppresses the expression of human KLF5 gene. With regard to siRNA Nos. 4, 8, and 10, human sequences corresponding thereto are completely the same as mouse sequences. Thus, it was considered that siRNA Nos. 8 and 10 suppress not only mouse KLF5 gene but also human KLF5 gene, as in the case of siRNA No. 4.

TABLE 2

Human KLF5 cDNA

SEQ

| | | |
|---|---|---|
| siRNA | Mouse KLF5 cDNA | ID |

| | | | | | |
|---|---|---|---|---|---|
| No. | Sequence | Position | Corresponding sequence | Position | No. |

| | | | | | |
|---|---|---|---|---|---|
| No. 2 | AAATTTACCTGCCACTCTGCC | 1156-1176 | AAATTTACCCACCACCCTGCC | 1334-1354 | 12 |
| No. 3 | AAGGAGTAACCCGGATCTGGA | 1216-1236 | AAGGAGTAACCCCGATTTGGA | 1394-1414 | 13 |
| No. 4 | AAAAGCTCACCTGAGGACTCA | 1303-1323 | AAAAGCTCACCTGAGGACTCA | 1481-1501 | 4 |
| No. 7 | AAATGGAGAAGTATCTGACCC | 405-425A | AAATGGAGAAGTATCTGACAC | 583-603 | 14 |
| No. 8 | AAAGTATAGACGAGACAGTGC | 463-483A | AAAGTATAGACGAGACAGTGC | 641-661 | 8 |
| No. 9 | AAACCAGACGGCAGTAATGGA | 874-894A | AAATCAGACAGCAGCAATGGA | 1040-1060 | 15 |
| No. 10 | AAGCTCAGAGCCTGGAAGTCC | 2048-2068 | AAGCTCAGAGCCTGGAAGTCC | 1226-1246 | 10 |
| No. 11 | AAGCCGTTCCAGTGCATGGTG | 1424-1444 | AAGCCCTTCCAGTGCGGGGTG | 1602-1622 | 16 |

### EXAMPLE 4

### Inhibition of Migration of Vascular Endothelial Cells by siRNA Capable of Suppressing Expression of KLF5 Gene

Inhibition of the migration of vascular endothelial cells by siRNA No. 4 capable of suppressing the expression of KLF5 gene was examined by performing an in vitro cell migration test on the vascular endothelial cells, using the following micropore filter (J. Cell Biol., 147, 1073-1084, 1999; Becton, Dickinson and Company, Technical Bulletin, 429, 1998).

Human umbilical cord venous endothelial cells (HUVEC; obtained from Sanko Junyaku Co., Ltd.; Production No. CC-2517) were inoculated into a 6-cm dish (Corning), resulting in a concentration of approximately 3×10⁵ cells. 10 μl of a cell transfection reagent (Lipofectamine 2000, manufactured by Invitrogen) was added to each of 200 μmol siRNA No. 4 and SEAP-siRNA used as a control, and then mixed. The obtained mixture was incubated at room temperature for 20 minutes, and the total amount thereof was then added to each dish. Thereafter, the cells were incubated in the presence of 5% CO₂ at 37° C. for 18 hours, so as to transfect each siRNA into the cells.

Thereafter, siRNA-transfected cells were washed, and the cells were then fluorescently labeled with 5 μg/ml fluorescent dye for staining living cells (Calcein A M, manufactured by Dojindo Laboratories). The thus obtained fluorescently labeled cells were peeled off with trypsin and then washed. Thereafter, the resultant cells were resuspended in a basal medium for vascular endothelial cells (EBM-2, manufactured by Sanko Junyaku Co., Ltd.), resulting in a cell concentration of 5×10⁵ cells/mi. Individual HTS FluoroBlok Inserts (inserts for 24-well plates; 3 μm pore size; BD Falcon) were attached to a 24-well cell culture insert plate (BD Falcon). Thereafter, 100 μl of a suspension containing the fluorescently labeled cells was added to the inserts, and 600 μl of a growth medium for vascular endothelial cells (Bullet kit EGM-2, manufactured by Sanko Junyaku Co., Ltd.) which contained 10 ng/ml human VEGF (manufactured by R & D Systems Inc.), was added to the 24-well plate.

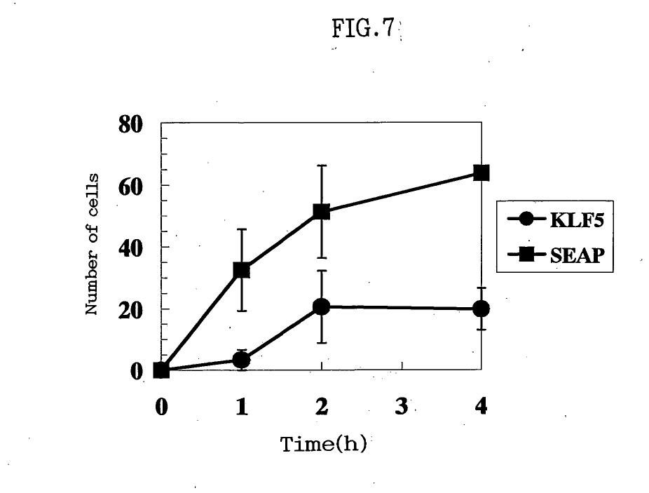

The cells which migrated through micropores of a filter, were observed and photographed by a fluorescence microscope from the bottom of the plate over time, up to 4 hours after the addition. The number of migrated cells was counted from the obtained images by using image analysis software (manufactured by Scion Image, Scion). As shown in FIG. 7, the number of migrated cells in vascular endothelial cells transfected with KLF5 gene-specific siRNA No. 4, was smaller than that in vascular endothelial cells transfected with SEAP-siRNA used as a control. Thus, it was confirmed that siRNA capable of suppressing the expression of KLF5 gene is able to inhibit the migration of vascular endothelial cells.

### EXAMPLE 5

### In vivo Angiogenesis-Inhibiting Effect of siRNA Capable of Suppressing the Expression of KLF5 Gene

The in vivo angiogenesis-inhibiting effect of siRNA No.4 capable of suppressing the expression of KLF5 gene was examined by an assay using the Matrigel as shown below (Proc. Natl. Acad. Sci. USA, 94, 13612-13617, 1997; J. Biol. Chem., 277, 6667-6675, 2002).

A Matrigel mixture was prepared by adding 0.6 μg of mouse VEGF (manufactured by R & D Systems Inc.; Catalog No. 493-MV), 0.6 μg of bovine basic fibroblast growth factor (bFGF; manufactured by R & D Systems Inc.; Catalog No. 133-FB), and 10 μg of siRNA No. 4, to 0.5 ml (5 mg) of Matrigel Matrix (manufactured by BD Bioscience), and then mixing them via pipetting on ice. As a control, a Matrigel mixture containing SEAP-siRNA instead of siRNA No. 4 was also prepared. The thus prepared Matrigel mixture was subcutaneously injected into the back of a 6-week-old male C57BL/6 mouse. Fourteen days after the injection, the gelatinized Matrigel was removed therefrom. The obtained Matrigel was washed with PBS once and then immobilized with a 10% formaldehyde-PBS solution. The immobilized Matrigel was sliced into pieces with a thickness of 5 mm, and then embedded in a paraffin. Thereafter, the Matrigel sections were made by common histological means and then stained with hematoxylin and eosin. The stained Matrigel sections were observed under a microscope.

As a result, in the Matrigel to which SEAP-siRNA as a control had been added, a large number of vascular endothelial cells reacted with VEGF and bFGF added to the Matrigel, migrated, and infiltrated in Matrigel. In contrast, in the Matrigel to which siRNA No. 4 had been added, infiltration of vascular endothelial cells into the Matrigel was suppressed. Thus, it was confirmed that siRNA capable of suppressing the expression of KLF5 gene is able to inhibit angiogenesis.

### EXAMPLE 6

### In vivo Antitumor Effect of siRNA that Suppresses Expression of KLF5 Gene

The in vivo antitumor effect of siRNA No. 4 capable of suppressing the expression of KLF5 gene was examined using suppression of the growth of tumor as an indicator as follows.

The mouse Lewis lung carcinoma cell line LL/2 (obtained from Dainippon Pharmaceutical Co., Ltd.; Catalog No. 09-1642) was subcutaneously injected into the back of a 5-week-old male C57BL/6 mouse at a concentration of 1×10⁶ cells. Two days after the injection, immobilization of the Lewis lung carcinoma was confirmed, and siRNA No. 4 was then subcutaneously injected into the periphery of the cancer. As a control, SEAP-siRNA was also subcutaneously injected into the periphery of the cancer. With regard to the dosage of siRNA No. 4 and that of SEAP-siRNA, 1 μg of siRNA or SEAP-siRNA was dissolved in 50 μl of water for injection (Otsuka distilled water, manufactured by Otsuka Pharmaceutical Co., Ltd.), and the obtained solution was used per mouse. The above siRNA was administered to the mouse for 8 consecutive days. The number of administrations was once a day. The tumor volume after administration was calculated using the following formula (1), and an increase in the tumor volume was compared with the case of the control.
tumor volume (mm³)={tumor length (mm)×tumor width (mm)²}/2 Formula (1):

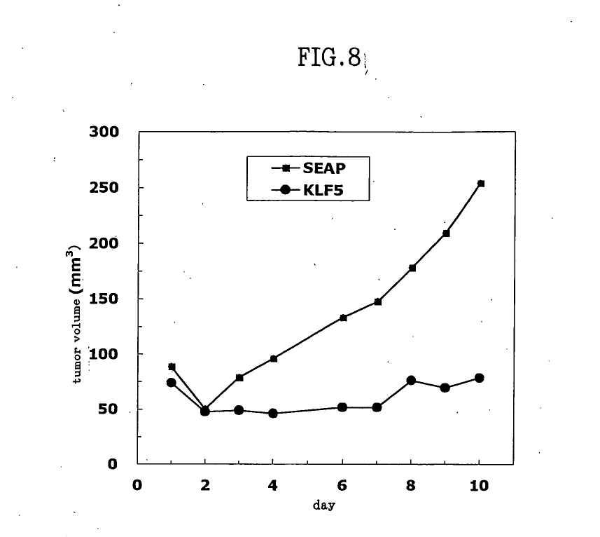

As a result, as shown in FIG. 8, the tumor volume of a mouse to which SEAP-siRNA as a control had been administered increased after initiation of the administration thereof. In contrast, an increase in the tumor volume of a mouse to which siRNA No. 4 had been administered was suppressed from one day after the administration thereof. Thus, it was confirmed that siRNA capable of suppressing the expression of KLF5 gene has an in vivo antitumor effect, and that the growth of tumor can be suppressed by the administration thereof.

“Sequence Listing Free Text”

| | |
|---|---|
| SEQ ID NO: 1 - | Inventors: Ryozo Nagai; Ichiro Manabe; |
| | Atsushi Ishihara |
| | Inventor: Tsuneaki Tottori |
| SEQ ID NO: 17 | siRNA No. 1 sense strand |
| SEQ ID NO: 18 | siRNA No. 1 antisense strand |
| SEQ ID NO: 19 | siRNA No. 2 sense strand |
| SEQ ID NO: 20 | siRNA No. 2 antisense strand |
| SEQ ID NO: 21 | siRNA No. 3 sense strand |
| SEQ ID NO: 22 | siRNA No. 3 antisense strand |
| SEQ ID NO: 23 | siRNA No. 4 sense strand |
| SEQ ID NO: 24 | siRNA No. 4 antisense strand |
| SEQ ID NO: 25 | siRNA No. 5 sense strand |
| SEQ ID NO: 26 | siRNA No. 5 antisense strand |
| SEQ ID NO: 27 | siRNA No. 6 sense strand |
| SEQ ID NO: 28 | siRNA No. 6 antisense strand |
| SEQ ID NO: 29 | siRNA No. 7 sense strand |
| SEQ ID NO: 30 | siRNA No. 7 antisense strand |
| SEQ ID NO: 31 | siRNA No. 8 sense strand |
| SEQ ID NO: 32 | siRNA No. 8 antisense strand |
| SEQ ID NO: 33 | siRNA No. 9 sense strand |
| SEQ ID NO: 34 | siRNA No. 9 antisense strand |
| SEQ ID NO: 35 | siRNA No. 10 sense strand |
| SEQ ID NO: 36 | siRNA No. 10 antisense strand |
| SEQ ID NO: 37 | siRNA No. 11 sense strand |
| SEQ ID NO: 38 | siRNA No. 12 antisense strand |
| SEQ ID NO: 39 | siRNA-SEAP sense strand |
| SEQ ID NO: 40 | siRNA-SEAP antisense strand |
| SEQ ID NO: 41 | KLF5 gene-specific forward primer |
| SEQ ID NO: 42 | KLF5 gene-specific reverse primer |
| SEQ ID NO: 43 | PDGF-A gene-specific forward primer |
| SEQ ID NO: 44 | PDGF-A gene-specific reverse primer |
| SEQ ID NO: 45 | SMemb gene-specific forward primer |
| SEQ ID NO: 46 | SMemb gene-specific reverse primer |
| SEQ ID NO: 47 | SRF gene-specific forward primer |
| SEQ ID NO: 48 | SRF gene-specific reverse primer |

1. An RNA capable of suppressing the expression of KLF5 gene, which comprises a sequence consisting of 15 to 30 contiguous nucleotides of KLF5 mRNA and a sequence complementary to the sequence.

2. The RNA according to claim 1, wherein the KLF5 mRNA is human KLF5 mRNA or mouse KLF5 mRNA.

3. The RNA according to claim 1, wherein the RNA is a double-stranded RNA consisting of a strand of a sequence consisting of 15 to 30 contiguous nucleotides of KLF5 mRNA and a strand of a sequence complementary to the sequence, in which 1 to 6 nucleotides are added to the 3′-terminus of each of the strands.

4. The RNA according to claim 1, wherein the RNA is an RNA forming a hairpin structure, which is obtained by ligating an RNA having a sequence consisting of 15 to 30 contiguous nucleotides of the KLF5 mRNA to an RNA having a sequence complementary to the sequence via a spacer oligonucleotide, and then adding 1 to 6 nucleotides to the 3′-terminus thereof.

5. An RNA capable of suppressing the expression of KLF5 gene, which is selected from the group consisting of the following (a) to (c):
(a) a double-stranded RNA having a strand of a sequence shown in any one of SEQ ID NOS: 2 to 16 and a strand of a sequence complementary to the sequence, in which 2 to 4 uridylic acids or deoxythymidylic acids are added to the 3′-terminus of each of the strands;
(b) an RNA forming a hairpin structure, which is obtained by ligating an RNA having a sequence shown in any one of SEQ ID NOS: 2 to 16 to an RNA having a sequence complementary to the sequence via spacer oligonucleotide that has 2 uridylic acids or deoxythymidylic acids at the 5′-terminus thereof, and then adding 2 to 4 uridylic acids or deoxythymidylic acids to the 3′-terminus thereof; and
(c) a double-stranded RNA consisting of a strand of a sequence shown in any one of SEQ ID NOS: 2 to 11 and a strand of a sequence complementary to the sequence, in which 2 uridylic acids are added to the 3′-terminus of each of the strands.

6. A vector, which allows the RNA according to claim 1 to be expressed.

7. A method of suppressing the expression of KLF5 gene in cells by transfecting the RNA according to claim 1 into the cells.

8. A method of suppressing the expression of a gene whose transcription is activated by KLF5 in cells by transfecting the RNA according to claim 1 into the cells.

9. The method according to claim 8, wherein the gene whose transcription is activated by KLF5 is platelet-derived growth factor A chain gene or a smooth muscle myosin heavy chain SMemb gene.

10. A pharmaceutical composition, which comprises, as an active ingredient, the RNA according to claim 1.

11. A pharmaceutical composition for inhibiting angiogenesis, which comprises, as an active ingredient, the RNA according to claim 1.

12. A therapeutic or preventive agent for cardiovascular disease or cancer, which comprises, as an active ingredient, the RNA according to claim 1.

13. The therapeutic or preventive agent according to claim 12, wherein the cardiovascular disease is arteriosclerosis, restenosis occurring after coronary intervention, or cardiac hypertrophy.

14. A method of suppressing the expression of KLF5 gene in cells by transfecting the vector according to claim 6 into the cells.

15. A method of suppressing the expression of a gene whose transcription is activated by KLF5 in cells by transfecting the vector according to claim 6 into the cells.

16. A pharmaceutical composition, which comprises, as an active ingredient, the vector according to claim 6.

17. A pharmaceutical composition for inhibiting angiogenesis, which comprises, as an active ingredient, the vector according to claim 6.

18. A therapeutic or preventive agent for cardiovascular disease or cancer, which comprises, as an active ingredient, the vector according to claim 6.


SEQUENCE LISTING (52 sequences)

SEQ ID NO: 1 - RNA, 19 nt, Mus musculus
caugaacgucuuccucccu

SEQ ID NO: 2 - RNA, 19 nt, Mus musculus
auuuaccugccacucugcc

SEQ ID NO: 3 - RNA, 19 nt, Mus musculus
ggaguaacccggaucugga

SEQ ID NO: 4 - RNA, 19 nt, Mus musculus
aagcucaccugaggacuca

SEQ ID NO: 5 - RNA, 19 nt, Mus musculus
uccccagaccguccaugcc

SEQ ID NO: 6 - RNA, 19 nt, Mus musculus
cgcugcgcccacccgccug

SEQ ID NO: 7 - RNA, 19 nt, Mus musculus
auggagaaguaucugaccc

SEQ ID NO: 8 - RNA, 19 nt, Mus musculus
aguauagacgagacagugc

SEQ ID NO: 9 - RNA, 19 nt, Mus musculus
accagacggcaguaaugga

SEQ ID NO: 10 - RNA, 19 nt, Mus musculus
gcucagagccuggaagucc

SEQ ID NO: 11 - RNA, 19 nt, Mus musculus
gccguuccagugcauggug

SEQ ID NO: 12 - RNA, 19 nt, Homo sapiens
auuuacccaccacccugcc

SEQ ID NO: 13 - RNA, 19 nt, Homo sapiens
ggaguaaccccgauuugga

SEQ ID NO: 14 - RNA, 19 nt, Homo sapiens
auggagaaguaucugacac

SEQ ID NO: 15 - RNA, 19 nt, Homo sapiens
aucagacagcagcaaugga

SEQ ID NO: 16 - RNA, 19 nt, Homo sapiens
gcccuuccagugcggggug

SEQ ID NO: 17 - DNA, 21 bp, Artificial Sequence
  Description of Combined DNA/RNA Molecule Synthetic oligonucleotide
  Description of Artificial Sequence Synthetic oligonucleotide
caugaacgucuuccucccutt

SEQ ID NO: 18 - DNA, 21 bp, Artificial Sequence
  Description of Combined DNA/RNA Molecule Synthetic oligonucleotide
  Description of Artificial Sequence Synthetic oligonucleotide
agggaggaagacguucaugtt

SEQ ID NO: 19 - RNA, 21 nt, Artificial Sequence
  Description of Artificial Sequence Synthetic oligonucleotide
auuuaccugccacucugccuu

SEQ ID NO: 20 - RNA, 21 nt, Artificial Sequence
  Description of Artificial Sequence Synthetic oligonucleotide
ggcagaguggcagguaaauuu

SEQ ID NO: 21 - RNA, 21 nt, Artificial Sequence
  Description of Artificial Sequence Synthetic oligonucleotide
ggaguaacccggaucuggauu

SEQ ID NO: 22 - RNA, 21 nt, Artificial Sequence
  Description of Artificial Sequence Synthetic oligonucleotide
uccagauccggguuacuccuu

SEQ ID NO: 23 - RNA, 21 nt, Artificial Sequence
  Description of Artificial Sequence Synthetic oligonucleotide
aagcucaccugaggacucauu

SEQ ID NO: 24 - RNA, 21 nt, Artificial Sequence
  Description of Artificial Sequence Synthetic oligonucleotide
ugaguccucaggugagcuuuu

SEQ ID NO: 25 - RNA, 21 nt, Artificial Sequence
  Description of Artificial Sequence Synthetic oligonucleotide
uccccagaccguccaugccuu

SEQ ID NO: 26 - RNA, 21 nt, Artificial Sequence
  Description of Artificial Sequence Synthetic oligonucleotide
ggcauggacggucuggggguu

SEQ ID NO: 27 - RNA, 21 nt, Artificial Sequence
  Description of Artificial Sequence Synthetic oligonucleotide
cgcugcgcccacccgccuguu

SEQ ID NO: 28 - RNA, 21 nt, Artificial Sequence
  Description of Artificial Sequence Synthetic oligonucleotide
caggcgggugggcgcagcguu

SEQ ID NO: 29 - RNA, 21 nt, Artificial Sequence
  Description of Artificial Sequence Synthetic oligonucleotide
auggagaaguaucugacccuu

SEQ ID NO: 30 - RNA, 21 nt, Artificial Sequence
  Description of Artificial Sequence Synthetic oligonucleotide
gggucagauacuucuccauuu

SEQ ID NO: 31 - RNA, 21 nt, Artificial Sequence
  Description of Artificial Sequence Synthetic oligonucleotide
aguauagacgagacagugcuu

SEQ ID NO: 32 - RNA, 21 nt, Artificial Sequence
  Description of Artificial Sequence Synthetic oligonucleotide
gcacugucucgucuauacuuu

SEQ ID NO: 33 - RNA, 21 nt, Artificial Sequence
  Description of Artificial Sequence Synthetic oligonucleotide
accagacggcaguaauggauu

SEQ ID NO: 34 - RNA, 21 nt, Artificial Sequence
  Description of Artificial Sequence Synthetic oligonucleotide
uccauuacugccgucuggcuu

SEQ ID NO: 35 - RNA, 21 nt, Artificial Sequence
  Description of Artificial Sequence Synthetic oligonucleotide
gcucagagccuggaaguccuu

SEQ ID NO: 36 - RNA, 21 nt, Artificial Sequence
  Description of Artificial Sequence Synthetic oligonucleotide
ggacuuccaggcucugagcuu

SEQ ID NO: 37 - RNA, 21 nt, Artificial Sequence
  Description of Artificial Sequence Synthetic oligonucleotide
gccguuccagugcaugguguu

SEQ ID NO: 38 - RNA, 21 nt, Artificial Sequence
  Description of Artificial Sequence Synthetic oligonucleotide
caccaugcacuggaacggcuu

SEQ ID NO: 39 - RNA, 21 nt, Artificial Sequence
  Description of Artificial Sequence Synthetic oligonucleotide
agggcaacuuccagaccauuu

SEQ ID NO: 40 - RNA, 21 nt, Artificial Sequence
  Description of Artificial Sequence Synthetic oligonucleotide
auggucuggaaguugcccuuu

SEQ ID NO: 41 - DNA, 20 bp, Artificial Sequence
  Description of Artificial Sequence Synthetic primer
ggttgcacaaaagtttatac

SEQ ID NO: 42 - DNA, 22 bp, Artificial Sequence
  Description of Artificial Sequence Synthetic primer
ggcttggcgcccgtgtgcttcc

SEQ ID NO: 43 - DNA, 22 bp, Artificial Sequence
  Description of Artificial Sequence Synthetic primer
ctccagcgactcttggagatag

SEQ ID NO: 44 - DNA, 22 bp, Artificial Sequence
  Description of Artificial Sequence Synthetic primer
ttcaggttggaggtcgcacatg

SEQ ID NO: 45 - DNA, 25 bp, Artificial Sequence
  Description of Artificial Sequence Synthetic primer
aatgcccgccagcagctggagcgac

SEQ ID NO: 46 - DNA, 25 bp, Artificial Sequence
  Description of Artificial Sequence Synthetic primer
gctccttatactgatccgcatgccg

SEQ ID NO: 47 - DNA, 25 bp, Artificial Sequence
  Description of Artificial Sequence Synthetic primer
tggcaccagtgtctgctactgtcag

SEQ ID NO: 48 - DNA, 25 bp, Artificial Sequence
  Description of Artificial Sequence Synthetic primer
gctgccctatcacagccatctggtg

SEQ ID NO: 49 - DNA, 1591 bp, Mus musculus
  CDS (167)..(1504)
ccgagcccaggagccccgatctccgtgcccgccttcgtgagcgtctggctgccggcccaggggtcccccgccgcggccccccgccgagtccgccgtcccgtgccagcccgagcgaggtgggatcgcgatcgctccgtgtcccgctcccgtaatccccagaccgtccatgcccacgcgggtgctgaccatgagcgcccgcctgggaccactgccccagccgccggccgcgcaggccgagcccgtgttcgcgcagctcaagccggtgctgggcgctgcgaacccggcccgcgacgcggcgctcttctccggagacgatctgaaacacgcgcaccaccacccgcctgcgccgccgccagccgctggcccgcgactgccctcggaggagctggtccagacaagatgtgaaatggagaagtatctgacccctcagctccctccagttccgataatttcagagcataaaaagtatagacgagacagtgcctcagtggtagaccagttcttcactgacactgaaggcataccttacagcatcaacatgaacgtcttcctccctgacatcactcacctgagaactggcctctacaaatcccagagaccatgcgtaacacagatcaagacagaacctgttaccattttcagccaccagagcgagtcgacggcccctcctcctcctccggcccccacccaggctctccccgagttcactagtatcttcagctcccaccagaccacagcgccaccacaggaggtgaacaatatcttcatcaaacaagaacttcctataccagatcttcatctctctgtcccttcccagcagggccacctgtaccagctgttgaatacaccggatctagacatgcccagttcgacaaaccagacggcagtaatggacacccttaatgtttctatggcaggccttaacccacacccctctgctgttccacagacgtcaatgaaacagttccagggcatgcccccttgcacgtacaccatgccaagtcagtttcttccacagcaggccacttattttcccccgtcaccaccaagctcagagcctggaagtcccgatagacaagctgagatgctgcagaatctcaccccacctccgtcctatgccgctacaattgcttccaaactggcgattcacaacccaaatttacctgccactctgccagttaattcgccaactctcccacctgtcagatacaacagaaggagtaacccggatctggagaagcgacgtatccacttctgcgattataatggttgcacaaaagtttatacaaagtcgtctcacttaaaagctcacctgaggactcatacgggcgagaagccctacaagtgcacctgggagggctgcgactggaggtttgcccggtcggatgagctgacccgccactacaggaagcacacgggcgccaagccgttccagtgcatggtgtgccaacgcagcttctcccgctccgaccacctcgcgctgcacatgaagcgccaccagaactgagcgagcgaacgctgcgcccacccgcctgacgccttgcagtccgctttgccatcctttaaaccgcagacctaacttcataaaaag

SEQ ID NO: 50 - DNA, 3359 bp, Homo sapiens
  CDS (312)..(1682)
ggtacgtgcgctcgcggttctctcgcggaggtcggcggtggcgggagcgggctccggagagcctgagagcacggtggggcggggcgggagaaagtggccgcccggaggacgttggcgtttacgtgtggaagagcggaagagttttgcttttcgtgcgcgccttcgaaaactgcctgccgctgtctgaggagtccacccgaaacctcccctcctccgccggcagccccgcgctgagctcgccgacccaagccagcgtgggcgaggtgggaagtgcgcccgacccgcgcctggagctgcgcccccgagtgcccatggctacaagggtgctgagcatgagcgcccgcctgggacccgtgccccagccgccggcgccgcaggacgagccggtgttcgcgcagctcaagccggtgctgggcgccgcgaatccggcccgcgacgcggcgctcttccccggcgaggagctgaagcacgcgcaccaccgcccgcaggcgcagcccgcgcccgcgcaggccccgcagccggcccagccgcccgccaccggcccgcggctgcctccagaggacctggtccagacaagatgtgaaatggagaagtatctgacacctcagcttcctccagttcctataattccagagcataaaaagtatagacgagacagtgcctcagtcgtagaccagttcttcactgacactgaagggttaccttacagtatcaacatgaacgtcttcctccctgacatcactcacctgagaactggcctctacaaatcccagagaccgtgcgtaacacacatcaagacagaacctgttgccattttcagccaccagagtgaaacgactgcccctcctccggccccgacccaggccctccctgagttcaccagtatattcagctcacaccagaccgcagctccagaggtgaacaatattttcatcaaacaagaacttcctacaccagatcttcatctttctgtccctacccagcagggccacctgtaccagctactgaatacaccggatctagatatgcccagttctacaaatcagacagcagcaatggacactcttaatgtttctatgtcagctgccatggcaggccttaacacacacacctctgctgttccgcagactgcagtgaaacaattccagggcatgcccccttgcacatacacaatgccaagtcagtttcttccacaacaggccacttactttcccccgtcaccaccaagctcagagcctggaagtccagatagacaagcagagatgctccagaatttaaccccacctccatcctatgctgctacaattgcttctaaactggcaattcacaatccaaatttacccaccaccctgccagttaactcacaaaacatccaacctgtcagatacaatagaaggagtaaccccgatttggagaaacgacgcatccactactgcgattaccctggttgcacaaaagtttataccaagtcttctcatttaaaagctcacctgaggactcacactggtgaaaagccatacaagtgtacctgggaaggctgcgactggaggttcgcgcgatcggatgagctgacccgccactaccggaagcacacaggcgccaagcccttccagtgcggggtgtgcaaccgcagcttctcgcgctctgaccacctggccctgcatatgaagaggcaccagaactgagcactgcccgtgtgacccgttccaggtcccctgggctccctcaaatgacagacctaactattcctgtgtaaaaacaacaaaaacaaaaaaaaaacaagaaaaccacaactaaaactggaaatgtatattttgtatatttgagaaaacagggaatacattgtattaataccaaagtgtttggtcattttaagaatctggaatgcttgctgtaatgtatatggctttactcaagcagatctcatctcatctcatgacaggcagccagtctcaacatgggtaaggggtgggggtgaaggggagtgtgtgcagcgtttttacctaggcaccatcatttaatgtgacagtgttcagtaaacaaatcagttggcaggcaccagaagaagaatggattgtatgtcaagattttacttggcattgagtagtttttttcaatagtaggtaattccttagagatacagtatacctggcaattcacaaatagccattgaacaaatgtgtgggtttttaaaaattatatacatatatgagttgcctatatttgctattcaaaattttgtaaatatgcaaatcagctttataggtttattacaagttttttaggattcttttggggaagagtcataattcttttgaaaataaccatgaatacacttacagttaggatttgtggtaaggtacctctcaacattaccaaaatcatttctttagagggaaggaataatcattcaaatgaactttaaaaaagcaaatttcatgcactgattaaaataggattattttaaatacaaaaggcattttatatgaattataaactgaagagcttaaagatagttacaaaatacaaaagttcaacctcttacaataagctaaacgcaatgtcatttttaaaaagaaggacttaggggtcgttttcacatatgacaatgttgcatttatgatgcagttttcaagtaccaaaacgttgaattgatgatgcagttttcatatatcgagatgttcgctcgtgcagtactgttggttaaatgacaatttatgtggattttgcatgtaatacacagtgagacacagtaattttatctaaattacagtgcagtttagttaatctattaatactgactcagtgtctgcctttaaatataaatgatatgttgaaaacttaaggaagcaaatgctacatatatgcaatataaaatagtaatgtgatgctgatgctgttaaccaaagggcagaataaataagcaaaatgccaaaaggggtcttaattgaaatgaaaatttaattttgtttttaaaatattgtttatctttatttattttgtggtaatatagtaagtttttttagaagacaattttcataacttgataaattatagttttgtttgttagaaaagttgctcttaaaagatgtaaatagatgacaaacgatgtaaataattttgtaagaggcttcaaaatgtttatacgtggaaacacacctacatgaaaagcagaaatcggttgctgttttgcttctttttccctcttatttttgtattgtggtcatttcctatgcaaataatggagcaaacagctgtatagttgtagaattttttgagagaatgagatgtttatatattaacgacaattttttttttggaaaataaaaagtgcctaaaagaaaaaaaaaaaaaaaaaa

SEQ ID NO: 51 - protein, 446 aa, Mus musculus
MPTRVLTMSARLGPLPQPPAAQAEPVFAQLKPVLGAANPARDAALFSGDDLKHAHHHPPAPPPAAGPRLPSEELVQTRCEMEKYLTPQLPPVPIISEHKKYRRDSASVVDQFFTDTEGIPYSINMNVFLPDITHLRTGLYKSQRPCVTQIKTEPVTIFSHQSESTAPPPPPAPTQALPEFTSIFSSHQTTAPPQEVNNIFIKQELPIPDLHLSVPSQQGHLYQLLNTPDLDMPSSTNQTAVMDTLNVSMAGLNPHPSAVPQTSMKQFQGMPPCTYTMPSQFLPQQATYFPPSPPSSEPGSPDRQAEMLQNLTPPPSYAATIASKLAIHNPNLPATLPVNSPTLPPVRYNRRSNPDLEKRRIHFCDYNGCTKVYTKSSHLKAHLRTHTGEKPYKCTWEGCDWRFARSDELTRHYRKHTGAKPFQCMVCQRSFSRSDHLALHMKRHQN

SEQ ID NO: 52 - protein, 457 aa, Homo sapiens
MATRVLSMSARLGPVPQPPAPQDEPVFAQLKPVLGAANPARDAALFPGEELKHAHHRPQAQPAPAQAPQPAQPPATGPRLPPEDLVQTRCEMEKYLTPQLPPVPIIPEHKKYRRDSASVVDQFFTDTEGLPYSINMNVFLPDITHLRTGLYKSQRPCVTHIKTEPVAIFSHQSETTAPPPAPTQALPEFTSIFSSHQTAAPEVNNIFIKQELPTPDLHLSVPTQQGHLYQLLNTPDLDMPSSTNQTAAMDTLNVSMSAAMAGLNTHTSAVPQTAVKQFQGMPPCTYTMPSQFLPQQATYFPPSPPSSEPGSPDRQAEMLQNLTPPPSYAATIASKLAIHNPNLPTTLPVNSQNIQPVRYNRRSNPDLEKRRIHYCDYPGCTKVYTKSSHLKAHLRTHTGEKPYKCTWEGCDWRFARSDELTRHYRKHTGAKPFQCGVCNRSFSRSDHLALHMKRHQN